(19) United States
(12) Patent Application Publication
(10) Pub. No.: US 2009/0312823 A1
(43) Pub. Date: Dec. 17, 2009

(54) Heating and cooling pad, control unit therefor, system and method

(76) Inventors: Theodore Arthur Patience, Laguna Niguel, CA (US); Mehrdad Haghi, Laguna Niguel, CA (US)

Correspondence Address: CONNORS ASSOCIATES, 1600 DOVE ST, SUITE 220, NEWPORT BEACH, CA, 92660, US

(21) Appl. No.: 12/491,980

(22) Filed: Jun. 25, 2009

Related U.S. Application Data
(63) Continuation-in-part of application No. PCT/IB2007/004595, filed on Dec. 24, 2007.
(60) Provisional application No. 60/877,098, filed on Dec. 26, 2006.

(51) Int. Cl. A61F 7/08; F25B 21/02; F25B 29/00; G05D 23/00

(52) U.S. Cl. 607/104; 62/3.3; 165/253; 607/114; 700/300

(57) ABSTRACT

A personal, portable heating and cooling system for an individual includes a thin, flexible pad and a control unit in fluid communication with the pad for regulating the temperature of a heat transfer fluid flowing between the pad and the control unit. The pad has an internal chamber for holding the heat transfer fluid. The chamber is partitioned to form therein a passageway that directs the heat transfer fluid to flow there through along a circuitous path. The control unit has a housing enclosing a reservoir for holding the heat transfer fluid, a pump for pumping the heat transfer fluid between the reservoir and the internal chamber of the pad, and a heating and cooling apparatus for regulating the temperature of a heat transfer fluid flowing between the reservoir and the chamber. The control unit includes an electronic control circuit having a user interface that enables a user increase and decrease the temperature of the fluid. An alternate embodiment enables the user to select a temperature of the fluid and still another embodiment where the temperature varies over time in accordance with an individualized custom temperature profile.

This application is continuation-in-part application of PCT Application No. PCT/IB2007/004595, filed Dec. 24, 2007, entitled “Heating and Cooling Pad, Control unit Therefor, System and Method,” which claims the benefit under 35 USC 119(e) of U.S. Provisional Patent Application No. 60/877,098, entitled “Thermal Electric Heating And Cooling Liquid Circulating Pad,” filed Dec. 26, 2006. This related application is incorporated herein by reference and made a part of this application. If any conflict arises between the disclosure of the invention in this PCT application and that in the related provisional application, the disclosure in this utility application shall govern. Moreover, any and all U.S. patents, U.S. patent applications, and other documents, hard copy or electronic, cited or referred to in this application are incorporated herein by reference and made a part of this application.

### DEFINITIONS

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

The word “circuitous” means indirect, winding or meandering.

The word “rectangular” includes square.

### BACKGROUND

This background discussion is not intended to be an admission of prior art.

Many individuals use a heating pad for comfort, or as therapy for pain. Many conventional heating pads comprise a pad with wires impeded therein through which electrical current flows to heat the pad. Moreover, particularly when sleeping, the temperature tends to rise, especially when the individual user is covered by a blanket or other bedding and the heating pad is under the user. Thus, the problem which such conventional heating pads is that they are uncomfortable to lie upon and provide only heating. One way to both heat and cool is to employ a variable voltage power supply to enable a user to set the temperature of a “heating and cooling” pad. In general, however, commercially available variable voltage power supplies are expensive.

### SUMMARY

Our heating and cooling pad, control unit therefor, heating and cooling system, and method of controlling temperature while in bed has one or more of the features depicted in the embodiments discussed in the section entitled “DETAILED DESCRIPTION OF SOME ILLUSTRATIVE EMBODIMENTS.” The claims that follow define our pad, control unit therefor, system, and method, distinguishing them from the prior art; however, without limiting the scope of our pad, control unit therefor, system, and method, as expressed by these claims, in general terms, one or more, but not necessarily all, of their features are:

One, our heating and cooling system is lightweight, compact, portable, and may be operated at ambient air temperatures substantially from 41° F. to 122° F. It comprises a pad and control unit adapted to be placed in fluid communication with the pad. The control unit includes a housing having a reservoir for holding a heat transfer fluid and a fluid plumbing network for circulating the heat transfer fluid between an internal chamber of the pad and the reservoir. The fluid pressure within the chamber is at a predetermined level so the pad is comfortable to lie upon, for example, the fluid pressure within the chamber may be substantially from 3 to 20 pounds per square inch (psi). As directed by the user interacting with a user interface of the control unit, a bi-directional heat pump cools or heats the heat transfer fluid as the heat transfer fluid is circulated. The bi-directional heat pump may comprise a thermoelectric unit having a heating mode and cooling mode and through which the heat transfer fluid flows upon being circulated by a fluid pump between an inlet and an outlet of the pad's chamber.

Two, a suitable thermoelectric unit is solid-state device utilizing the “Peltier” effect. Such a solid-state device has a first side in heat transfer contact with the ambient air environment and a second side. An outlet of the pad is in communication with the thermoelectric unit so fluid flowing from the pad passes the second side and makes heat transfer contact with this second side. The thermoelectric unit is responsive to alternating the polarity of an applied voltage so heat flows from the second side into the fluid passing thereby or heat flows from the fluid passing thereby into the second side. By alternating the polarity of the applied voltage the fluid temperature is increased or decreased, and depending on the type of user interface employed, the user may select a specific fluid temperature.

Three, a control circuit for operating the thermoelectric unit and heat transfer pump within the housing is coupled to the user interface. In one embodiment, the user interface enables the user to simply increase or decrease the temperature of the heat transfer fluid flowing through the chamber, but not select a specific temperature. In another embodiment, the user interface enables the user to select a specific temperature. In still another embodiment, the user interface enables the user to create a program in which a plurality of different temperatures are selected and selectively applied over a period of time. In other words, the user may create his or her own individualized custom temperature profile of the heat transfer fluid over a selected period of time. These temperature profiles may be varied for therapeutic purposes (hot/cold therapy), or for the purpose of prompting comfortable sleep, for example, by having the pad be warmer at the beginning of the sleep cycle, then cooling down during the middle of the night, varying temperature in a controlled manner over time.

In these later embodiments enabling the user to select specific temperatures, at least one temperature sensor is used to detect the temperature of the heat transfer fluid. A switching circuit switches the thermoelectric unit between the heating mode and cooling mode as determined by a comparison between a user selected set point temperature and the temperature of the heat transfer fluid as detected by the temperature sensor. A second temperature sensor may be used to detect ambient air temperature along with a microprocessor that controls the rate of increase or decrease in temperature in response to the ambient air temperature detected by the second temperature sensor.

Four, our system may employ means for detecting an abnormal and unsafe condition, and upon detection of such condition, turn off the thermoelectric unit, or turn off both the thermoelectric unit and the pump, and provide a signal to the user that an abnormal and unsafe condition exists. For example: Another temperature sensor may be used for detecting the temperature of the heat transfer fluid, thermoelectric unit and pump being turned off when this temperature sensor detects that temperature of the heat transfer fluid is not within predetermined, preset limits. A level sensor on the exterior of the control unit may be used for detecting the spatial orientation of the control unit, thermoelectric unit and pump being turned off when the level sensor detects that the control unit is tipped over. A fluid sensor may be used for detecting the amount of heat transfer fluid in the reservoir, the thermoelectric unit and pump being turned off when the fluid sensor detects insufficient heat transfer fluid in the reservoir.

Five, a low cost power supply provides a constant output voltage, for example, substantially from 12 to 24 volts, and its output is electrically coupled to a converting circuit that is interactive active with the user interface. The converting circuit has an operator element that enables the user to increase or decrease the fluid temperature by converting the constant output voltage to a selectable variable voltage. In one embodiment, the variable voltage power supply having a power rating substantially from 200 to 400 Watts over a 24 volt range, and the thermoelectric unit has a power rating substantially from 150 to 300 Watts over a 24 volt range.

Six, our pad is thin and flexible pad and its internal chamber is partitioned to form therein a passageway that directs the heat transfer fluid to flow along a circuitous path between the inlet and outlet. Our pad may have a length substantially from 12 to 48 inches, a width substantially from 12 to 36 inches, a thickness that does not exceed approximately ½ inch when the heat transfer fluid flowing through the chamber at said pressure, and it may be substantially rectangular. The chamber may have a volume substantially from 100 milliliters to 2 liters with the heat transfer fluid flowing through the chamber at the pressure from 3 to 20 psi. Our pad may comprise a pair of overlying, liquid impenetrable, flexible plastic sheets bonded together by (a) linear welds between facing surfaces of the overlying sheets to form within the chamber a passageway that directs the heat transfer fluid to flow along a circuitous path between the inlet and outlet, and (b) spot welds within the passageway to form fluid mixing or turbulent flow zones along the passageway. Each sheet may have substantially the same dimensions, the sheets may each have edges that are bonded along the edges to form the overall width and length dimensions of the chamber, which are only slightly less than the width and length dimensions of the sheets. The plurality of spot welds may be arranged in a predetermined pattern to form upon the pad being horizontally orientated a plurality of cushion pillows along the passageway for enhanced comfort as the heat transfer fluid flows through the pad at the predetermined pressure. For example, the number of spot welds per square inch of facing surfaces may be from 4 to 25 and the area of each individual spot weld may be substantially from 0.003 to 0.012 square inches. The spot welds may be arranged in a predetermined grid pattern comprising intersecting grid lines with individual spot welds located at intersections of the grid lines.

Our method of controlling temperature while in bed comprises the following steps:

(a) positioning on the bed an enlarged thin flexible heating and cooling pad having an internal chamber holding a heat transfer fluid, the pad having width and length dimensions sufficient so a substantial portion of a user's body contacts the pad directly or indirectly when lying on the pad, said pad having a maximum height of substantially ½ inch when the chamber is filled with the heat transfer fluid at a pressure substantially from 3 to 20 pounds per square,

(b) connecting the pad to a control unit having a housing enclosing a reservoir for holding the heat transfer fluid, a pump for pumping the heat transfer fluid between the reservoir and the pad, and a heating and cooling apparatus for regulating the temperature of a heat transfer fluid flowing between the reservoir and the chamber, said control unit including an electronic controller having a user interface that enables a user enables a user to increase or decrease the temperature of the fluid, and

(c) increasing or decreasing the temperature of the heat transfer fluid flowing through the pad.

The pad may be constructed of antimicrobial material, and insulated tubing may be used to connect the pad to the control unit.

These features are not listed in any rank order nor is this list intended to be exhaustive.

### DESCRIPTION OF THE DRAWING

Some embodiments of our pad, control unit therefor, system, and method, are discussed in detail in connection with the accompanying drawing, which is for illustrative purposes only. This drawing includes the following figures (Figs.), with like numerals indicating like parts:

FIG. 1 is a schematic diagram of one embodiment of our heating and cooling system.

FIG. 1A is a schematic diagram of another embodiment of our heating and cooling system.

FIG. 2A is a user interface used with the embodiment of our heating and cooling system illustrated in FIG. 1.

FIG. 2B is a user interface used with the embodiment of our heating and cooling system illustrated in FIG. 1A.

FIG. 2C is a user interface used with still another embodiment of our heating and cooling system similar to that depicted in FIG. 1A.

FIGS. 3A through 3G depicted different temperature profiles over time employing the user interface illustrated in FIG. 2C.

FIG. 4A is a rear perspective view of one embodiment of our control unit for our heating and cooling pad.

FIG. 4B is a bottom perspective view of our control unit shown in FIG. 4A.

FIG. 4C is an exploded perspective view of our control unit shown in FIG. 4A.

FIG. 5 is a top plan view of one embodiment our heating and cooling pad.

FIG. 5A is a cross-sectional view taken along line 5A-5A of FIG. 5.

FIG. 6 is a schematic diagram of a temperature control board shown in FIG. 1A.

FIG. 7A is a schematic diagram of the heat/cool logic for the circuit shown in FIG. 1A.

FIG. 7B is a schematic diagram of the safety board logic for the circuit shown in FIG. 1A.

FIG. 7C is a process flow chart of a program for a microprocessor used with the interface shown in FIG. 2B.

FIG. 7D is a process flow chart for the user interface shown in FIG. 2C.

FIG. 8A is a schematic cross-sectional front view of a thermoelectric unit used in our heating and cooling system.

FIG. 8B is a schematic side view of the thermoelectric unit depicted in FIG. 8A.

FIG. 8C is a top plan view of the thermoelectric unit depicted in FIG. 8A.

FIG. 8D is a cross-sectional view taken along line 8D-8D of FIG. 8A.

### DETAILED DESCRIPTION OF SOME ILLUSTRATIVE EMBODIMENTS

### General

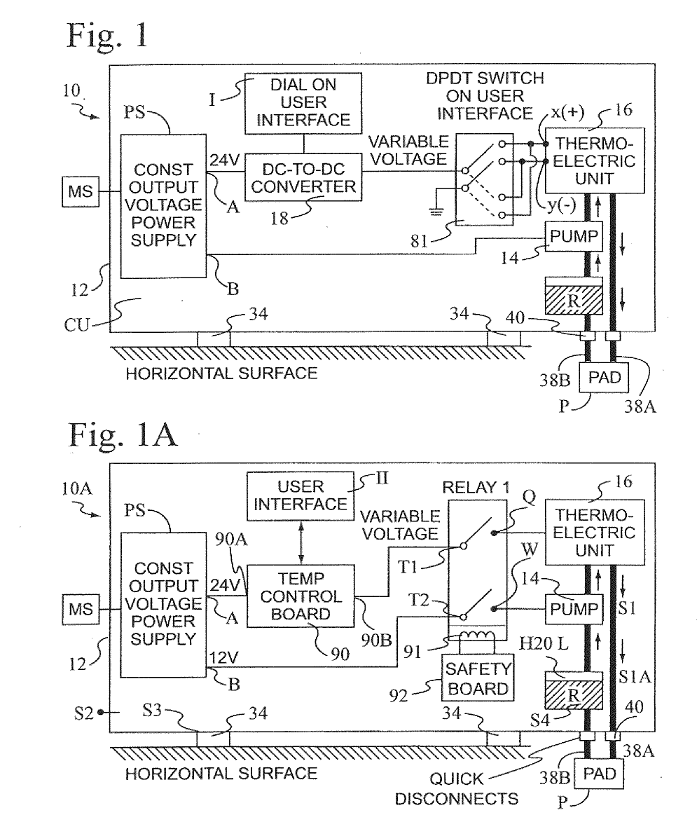

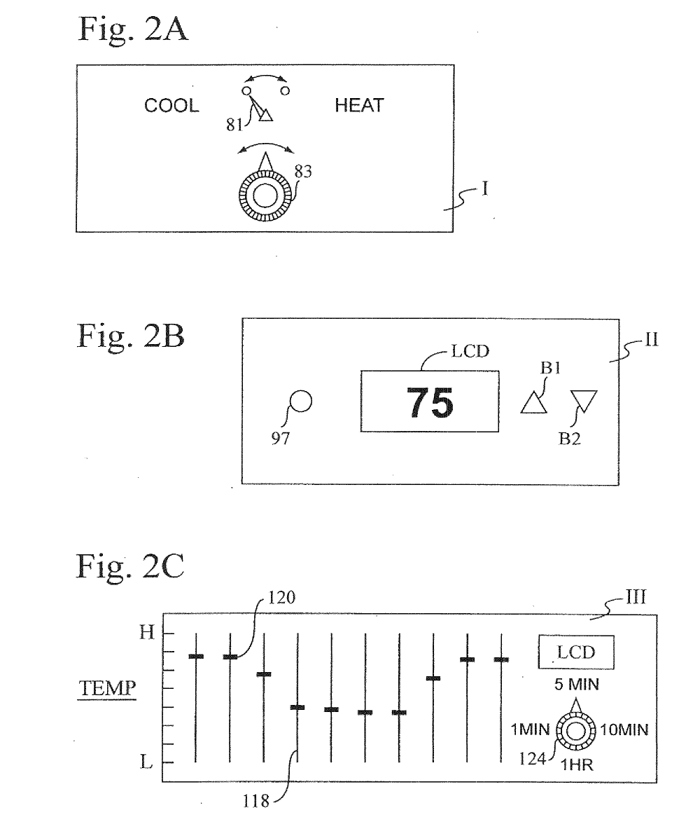

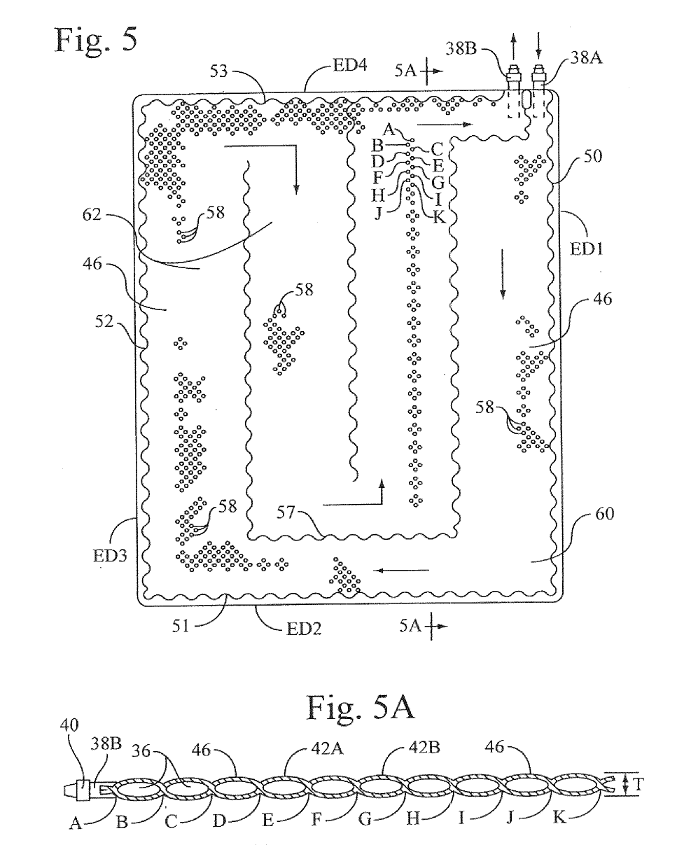

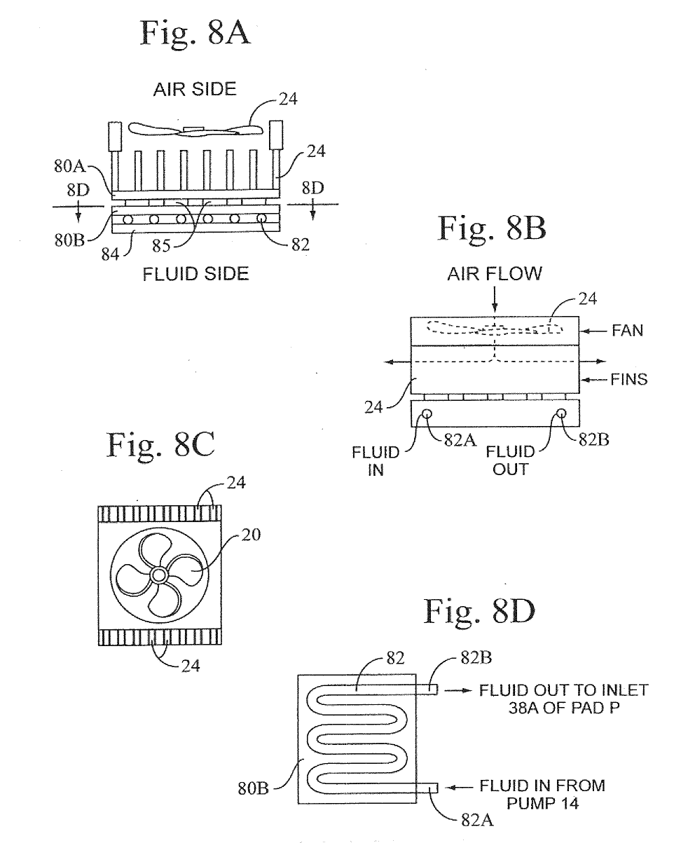

Our heating and cooling system comprises a pad P and a light-weight, portable, compact control unit CU having within it a bi-directional heat pump that either cools or heats a heat transfer fluid, for example, water, circulating between the control unit CU and the pad P. The control unit CU may use one of the interfaces I, II, and III, shown respectively in FIGS. 2A through 2C. These interfaces I, II or III each enable a user to select the temperature of the heat transfer fluid flowing through the pad P on which the user may recline. The pad P is lightweight, flexible, and with the heat transfer fluid flowing through it at a predetermined pressure, the pad is inflated so it is comfortable to lie upon. An example of one embodiment of our pad P is illustrated in FIGS. 5 and 5A. A plumbing network PN (FIGS. 1 and 1A) comprising any tubes, piping, conduits, connectors, couplers, etc. enabling the fluid to flow between the pad P and control unit CU directs the fluid to pass a side 80a (FIG. 8A) of a bi-directional heat pump that is in heat transfer contact with the fluid prior to the fluid returning to the pad P.

### FIGS. 1 & FIGS. 8A Through 8D

One embodiment of our heating and cooling system depicted in FIG. 1 is generally designated by the numeral 10, and its control unit CU employs the user interface I illustrated FIG. 2A. Regardless of the interface used, our control unit CU includes a housing 12 containing a pump 14, a thermoelectric unit 16, a reservoir R holding the heat transfer fluid, and a power supply PS. New Mark International, Inc. of Los Angeles, Calif. sells a suitable thermoelectric unit 16, which is a sold state device utilizing the “Peltier” effect and is operable over a range of temperatures substantially from 41° F. to 122° F., power ratings of 150 to 300 Watts, and voltages substantially from 0 to 24 volts. The pump 14 circulates the heat transfer fluid between the control unit CU and the pad P, and it may be mounted in a frame (not shown) with cushioned pads that insulate the housing 12 from pump vibrations and permit quieter operation. A relatively low cost, constant voltage power supply PS is used, for example, the power supply sold by Advanced Power Solutions of Pleasanton, Calif. This power supply PS has a constant 24-volt output at its terminal A and constant 12-volt output at its terminal B. Power is provided to the power supply PS is supplied through a main switch MS on the exterior of the housing 12.

Terminal A of the power supply PS is connected to a conventional DC-to-DC converting circuit 18 and may be obtained from Amest Corporation of Santa Margarita, Calif., a detail diagram of such converting circuit identified as #3 is shown in U.S. Provisional Patent Application No. 60/877,098, incorporated by reference herein. The circuit 18, which is discussed in greater detail subsequently, converts the constant 24 volt at the terminal A to a user selectable variable voltage, for example, a variable voltage from 0 to 24 volts, that is applied to the thermoelectric unit 16 through a DPDT switch 81. The position of the switch 81 determines the polarity being applied to terminals X and Y of the thermoelectric unit 16. With the switch 81 in the cool position in FIG. 2A and in solid lines in FIG. 1, the terminal X has a positive (+) polarity and the terminal Y has a negative (−) polarity. When the switch 81 in the heat position in FIG. 2A and in dotted lines in FIG. 1, the terminal X has a negative (−) polarity and the terminal Y has a positive (+) polarity. The dial 83 on the interface I enables the user to either increases or decrease the differential in voltage across the terminals X and Y by rotation in either a clockwise or counter-clockwise direction.

The thermoelectric unit 16 heats or cools the heat transfer fluid, functioning as the bi-directional heat pump. As illustrated in FIGS. 8A through 8D, the thermoelectric unit 16 has opposed sides 80a and 80b with a plurality of Peltier devices 85 in contact with these sides. The side 80a is in heat transfer contact with the ambient air environment that typically is at a temperature substantially from 41° F. to 122° F. The side 80b is in heat transfer contact with the heat transfer fluid flowing from the pad P. Specifically, a sinusoidal, corrosion resistance, copper tube 82 sandwiched between the side 80b and a support plate 84 has an influent end 82a in communication with the pump 14 which pumps the fluid from the pad P via the reservoir R into the tube 82. The thermoelectric unit 16 is mounted between the ambient air environment serving as a heat sink and that support plate 84 with brazed copper tube 82. The heat sink permits efficient heat transfer to the environment, while the plate and brazed copper tube 82 permits efficient heat transfer to and from the fluid. The plate and heat sink are separated from each other using a sheet of insulating foam to prevent direct heat conduction between the hot and cold sides of the thermoelectric unit. Depending on the position of the switch 81, the temperature of the heat transfer fluid in the pad P is either increased or decreased and the rate of increase or decrease is governed by the position of the dial 83. The fluid exits the tube 82 from its effluent end 82b that is in communication with the pad P. Thus, the heat transfer fluid flowing from the pad P and then through the reservoir R as it flows through the tube 82, passes the side 80b, making heat transfer contact with this side prior to returning to the inlet 38a of the pad P. Alternatively, instead of using the copper tube 82, a fluid chamber may be built which has the side 80b as a component of a structure having internal metallic walls that create a winding path through its interior. The fluid flows winding path and concurrently being in thermal contact with side 80b.

The thermoelectric unit 16 is responsive to alternating the polarity of the voltage applied to terminals X and Y and across the sides 80a and 80b. When the polarity of the applied voltage is as shown in solid lines as depicted in FIG. 1, heat flows from the fluid passing by the side 80b into the ambient air environment that serves as a heat sink. When the polarity of the applied voltage is as shown in dotted lines as depicted in FIG. 1, reversing the polarity at the terminals X and Y, heat flows into the fluid passing by the side 80b. A fan 20 constantly blows air past an array of fins 24 through which the heat transfer fluid flows as it circulates. The fan 20 increases or forces heat transfer, and it is operational when the thermoelectric unit 16 is operational. The power supply PS provides power directly for the fan 20 and pump 14, allowing the use of DC fans for quieter operation. The DC-to-DC converting circuit 18 controls the DC voltage and current being supplied to the thermoelectric unit 16, allowing continuously adjustability between the maximum and minimum temperatures the unit is designed to deliver based on user input.

### FIG. 1A

As depicted in FIG. 1A, an alternate embodiment of our heating and cooling system is generally designated by the numeral 10a, and its control unit CU employs the user interface II illustrated FIG. 2B that enables a user to select the temperature of the fluid flowing through the pad P. Our heating and cooling system 10a includes a plurality of sensors S1, S1A, S2, S3 and S4. The sensors S1 and S1A are temperature sensors located in the effluent stream exiting the thermoelectric unit 16 upstream of the outlet 38a. The sensor S2 is a temperature sensor located in the control unit CU that detects the temperature of the ambient air environment. The sensor S3 is a sensor that detects when the control unit CU is tipped and no longer resting on a horizontal surface. The fluid sensor S4 is a sensor associated with the reservoir R that detects when the reservoir is near empty.

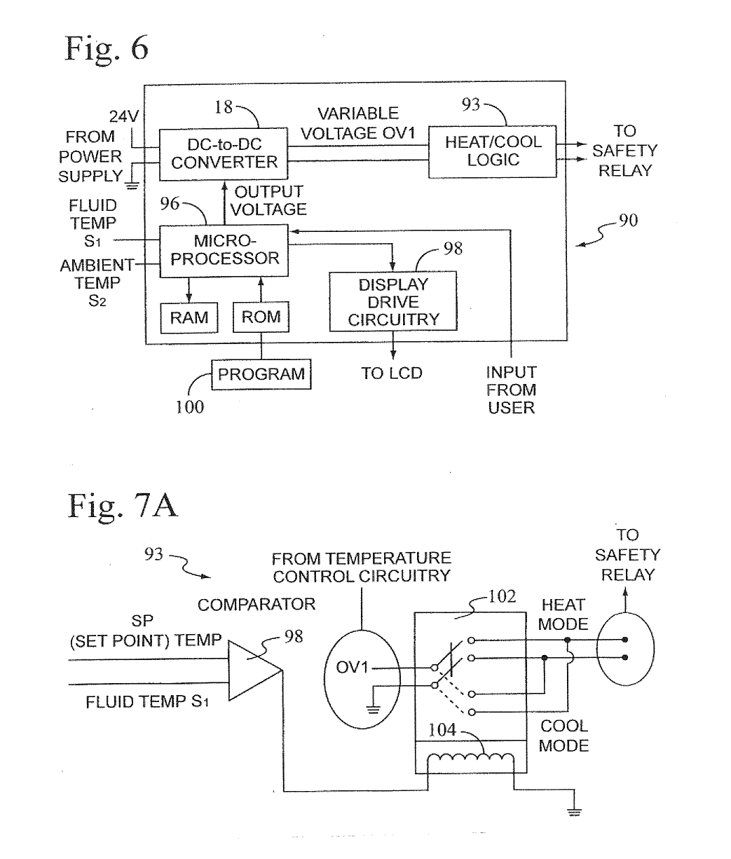

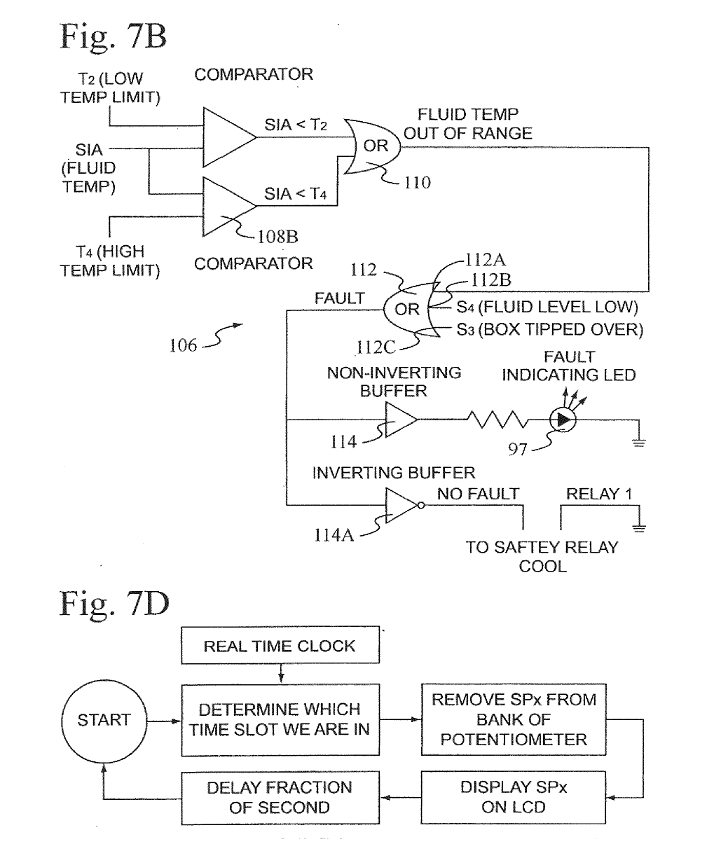

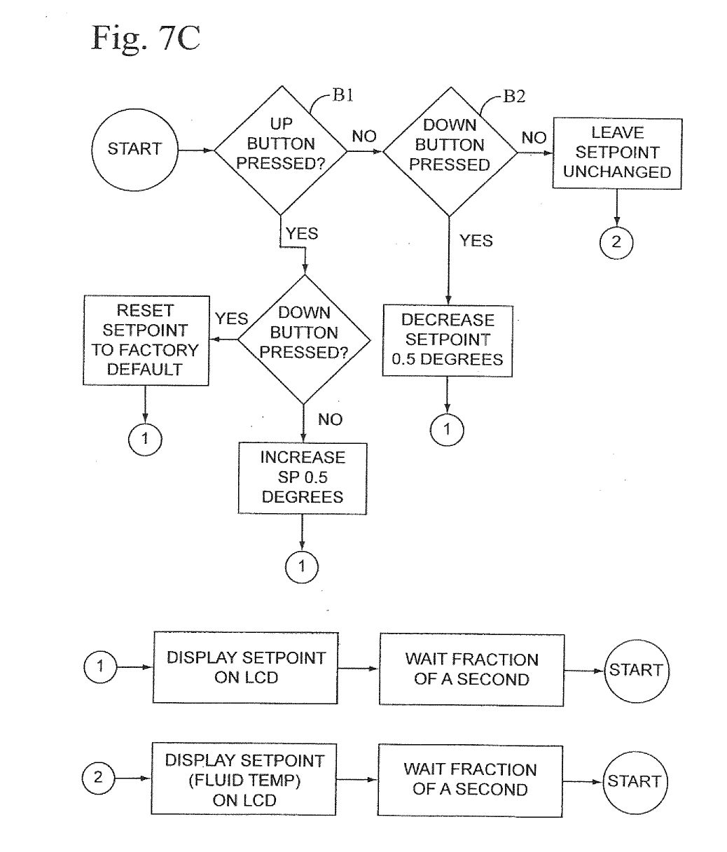

The control unit CU of our heating and cooling system 10a includes a temperature control board 90 illustrated in FIG. 6 and a safety board 92, and a failsafe, normally open Relay 1 that upon closing energizes the thermoelectric unit 16 and pump 14. The temperature control board 90 and its heat/cool logic circuit 93 depicted in FIG. 7A enables the user to set the temperature of the fluid circulating through our system 10a. The temperature control board 90 has an input 90a (FIG. 1A) connected to the 24 volt terminal A of the power supply PS and an output 90b connected to one terminal T1 of the Relay 1. The 12-volt terminal B of the power supply PS is connected to another terminal T2 of the Relay 1. The safety board 92 is coupled through a coil 91 to the Relay 1. One output Q from the Relay 1 is connected to the thermoelectric unit 16 and another output W from the Relay 1 is connected to the pump 14. The user sets the temperature of the fluid by actuating one or the other of a pair of buttons B1 and B2 of the interface II.

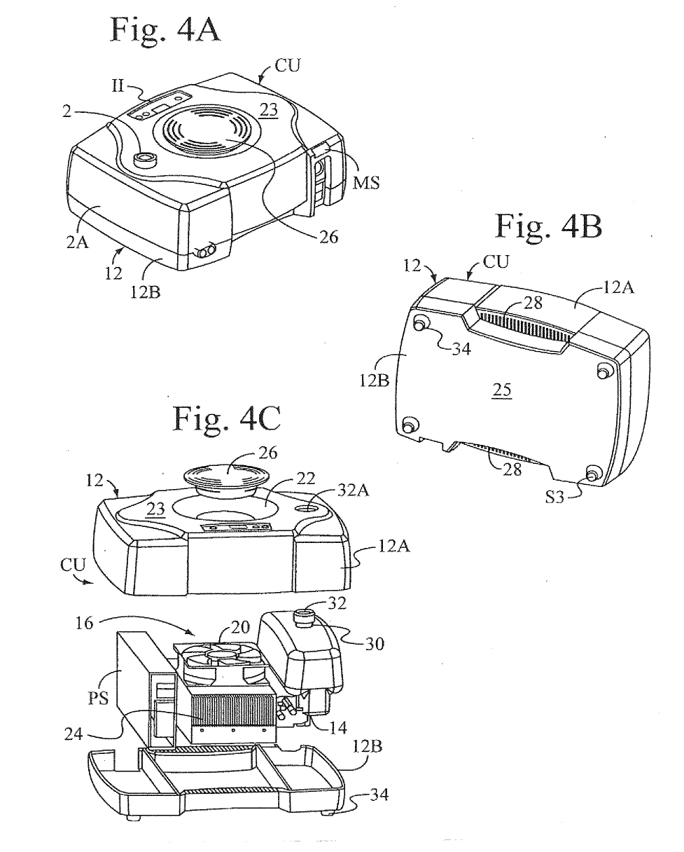

### FIGS. 4A Through 4C

The physical configuration of the control unit CU is designed so the control unit sits on a substantially horizontal surface, and thereby maintains the reservoir R in a substantially vertical orientation so the liquid heat transfer fluid does not easily spill. As illustrated in FIGS. 4A through 4C, the control unit's housing 12 comprises a molded plastic top section 12a and a molded plastic bottom section 12b that fit snug together to provide a block shaped or cubical console having an interior, which may be insulated, holding the power supply PS, the thermoelectric unit 16, the reservoir R, and the pump 14. The housing 12 is compact, with typical dimensions being a height substantially from 5.5 to 6.5 inches, a length substantially from 12 to 15 inches, and a width substantially from 9 to 12 inches. The thermoelectric unit 16 is positioned between the power supply PS and the reservoir R. Its fan 20 that is seated beneath an opening 22 in the top wall 23 to the top section 12a and above front and rear fins 24 through which the heat transfer fluid flows.

A removable slotted cap 26 fits snug within the opening 22. A pair of gratings 28 respectively at a lower front and lower back edge of the top section 12a allows air to flow through the slotted cap 26, past the fins 24, and out the gratings 28 when the fan 20 is operating. The reservoir R is a closed container with an access aperture 30 in its top that has a removable plug 32 protruding through a hole 32a in the top wall 23 for removal when the reservoir needs to be refilled with heat transfer fluid. As illustrated in FIG. 4B, the bottom section 12b has on its underside surface 25 at each of its corners a rubber cushion or foot element 34, which, as discussed subsequently in greater detail, may include the tip over indicator sensor S3 (FIG. 1A) The tip over indicator sensor S3 may be imbedded in a foot element 34 as shown in FIG. 4B.

### FIGS. 5 and 5A

As best depicted in FIGS. 5 and 5A, our pad P is thin, flexible, substantially rectangular in configuration, and it may be rolled up for storage. A sheet material having an exterior surface including an antimicrobial material is used to make the pad P. Both exposed surfaces of the pad P may contain the antimicrobial material. Micropel 5PVC is a suitable antimicrobial additive material provided by Lamcotec, Inc. of Corona, Calif. The active ingredient of Micropel 5PVC is 10, 10-Oxybisphenoxarsine (OBPA), and the OBPA (2-5%) is mixed with a polymeric resin carrier OBPA (95-98%). Micropel 5PVC is used in the conventional manner to make the exposed surfaces of the pad P resistant to the build up of bacteria.

The pad P allows the heat transfer fluid to pass through it without tubes or pipes or without being crimped by the weight of the person on the pad. It has an internal chamber 36 (FIG. 5A) for holding the heat transfer fluid and an inlet 38a and outlet 38b in fluid communication with the chamber. Water tight, quick connect-disconnect couplers 40 at the inlet and outlet prevent fluid leakage. As the heat transfer fluid flows through our pad P, the fluid fills the chamber 36 and inflates the pad. In an unrolled condition, the pad P has a length l (FIG. 1) substantially from 12 to 48 inches and a width w substantially from 12 to 36 inches. In an un-inflated state our pad P has a thickness t that does not exceed 1/32 inch. When inflated with fluid filling the chamber 36 at a pressure substantially from 3 to 20 pounds per square inch (psi), preferably from 4 to 6 psi, our pad P has a maximum thickness t (FIG. 5A) that does not exceed approximately ½ inch, and typically ranges substantially from ⅛ to ⅓ inch. The chamber 36 has a volume substantially from 100 milliliters to 2 liters with the heat transfer fluid flowing through the chamber at the designated pressure.

Referring to FIG. 5A, the pad P comprises a pair of liquid impenetrable, plastic sheets 42a and 42b welded together and partitioned to form between them a circuitous fluid passageway 44 within the chamber 36. A 200 denier urethane coated nylon sheet has been used and may be obtained from Plas-Tech, Inc. of Corona, Calif. Each sheet 42a and 42b is substantially rectangular, each sheet has substantially the same dimensions, the sheets each have edges ED1 through ED4 and are bonded along these edges to form the overall width and length dimensions of the chamber 36, which are only slightly less than the width and length dimensions of the sheets. The inlet 38a and the outlet 38b are next to each other along the side edge ED4 of the pad P and are in fluid communication with the passageway 44. The passageway 44 includes a plurality of cushion pillows 46 that are inflated as the heat transfer fluid flows through the pad P upon the pad being horizontally orientated and filled with the heat transfer fluid at the pressure substantially from 3 to 20 pounds psi. Excessively high pressure may open the welds, causing the fluid to leak from the pad P, and make the pad too stiff and uncomfortable. Excessively low pressure may result in a poor heat transfer to the user and cause the pad to be uncomfortable. Thus, maintaining the pressure substantially from 3 to 20 pounds psi is important.

Welding together the overlying plastic sheets 38a and 38b may be accomplished by heating selected portions of the contacting facing surface of the sheets. For example, there may be linear welds 50 through 57 that may be wavy, for example, sinusoidal, and a plurality of spot welds 58 in the body of the pad P. The linear welds 50 through 53 each project along a straight line that is adjacent an edge ED1, ED2, ED3, or ED4, as the case may b. The linear welds 54, 57 are within the body of the pad P, with the linear welds 54 and 57 at a right angle with respect to each other, the linear welds 56 and 57 at a right angle with respect to each other, and linear welds 53 and 55 at a right angle with respect to each other. This arrangement of the linear welds 50 through 57 forms within the body of the pad the circuitous passageway 44. The plurality of spot welds 58 are arranged in a predetermined pattern to form the cushion pillows 46 upon the pad being horizontally orientated and inflated with the fluid. The cushion pillows 46 provide fluid mixing zones along the passageway 44. The number of spot welds 58 per square inch of facing surfaces is substantially from 4 to 25, the area of an individual spot welds is substantially from 0.003 to 0.012 square inches, and the spot welds may be arranged in a predetermined grid pattern comprising intersecting grid lines 60 and 62 with individual spot welds located at intersections of the grid lines, for example, at the intersections a through k.

### FIGS. 6 Through 7D

In our system 10a a user sets the temperature of the heat transfer fluid to be within the range of substantially from 50° to 125° F. and is adapted for operation at ambient air temperatures substantially from 41° F. to 122° F. In our system 10a (a) the temperature sensors S1 and S1A detect the temperature of the heat transfer fluid, with the thermoelectric unit 16 and pump 14 being turned off when the temperature sensor S1A detects that the temperature of the heat transfer fluid is not within predetermined limits set at the factory when the system 10a is manufactured, (b) the sensor S2 detects ambient air temperature and feeds this information to the circuit shown in FIG. 7A, (c) the level sensor S3 on the exterior of the control unit CU detects the spatial orientation of the control unit, with the thermoelectric unit 16 and the pump 14 being turned off when the level sensor detects that the control unit is tipped over, and (c) the fluid sensor S4 detects the amount of heat transfer fluid in the reservoir R, with the thermoelectric unit 16 and pump being turned off when the fluid sensor detects insufficient heat transfer fluid in the reservoir R. These sensors S1 through S4 and their associate circuits as discussed herein subsequently provide means for detecting an abnormal and unsafe condition, and upon detection of such condition, turn off the thermoelectric unit 16, or turn off both the thermoelectric unit 16 and pump 14, and provide a signal to the user that an abnormal or unsafe condition exists, for example, by activating an audio alarm or a warning light 97 (FIG. 2B) on the user interface II.

As shown in FIG. 2B, the user interface II has a selection mechanism comprising a pair of temperature advancing buttons B1 and B2 that enables the user to select a set point temperature of the heat transfer fluid. The selected temperature is displayed by a liquid crystal display LCD on the user interface II. Pressing the button B1 increases the temperature and pressing the button B2 decreases the temperature. The actual temperature of the fluid in the effluent stream is normally displayed in the liquid crystal display LCD, but upon pressing one of the buttons, the selected set point temperature is momentarily displayed in the liquid crystal display LCD, for example, 75° F. As shown in FIG. 7A, a comparator 98 compares the user selected set point temperature with the fluid temperature detected by the temperature sensor S1. If required based on this comparison, the thermoelectric unit 16 is signaled to increase or decrease the temperature of the heat transfer fluid flowing between the reservoir R and the pad P. A microprocessor 96 (FIG. 6) governs the rate of increase or decrease the temperature of the heat transfer fluid as determined by the ambient temperature detected by the sensor S2. If the temperature sensor S1A detects that fluid temperature is outside the limits set during manufacture at the factory, the thermoelectric unit 16 and pump 14 are turned off and the warning light 97 is activated.

As illustrated in FIG. 6, the temperature control board 90 (FIG. 1A) is used to enable the user to select the temperature of the heat transfer fluid flowing through the pad P. This board includes the DC-to-DC converting circuit 18, the heat/cool logic circuit 93, a microprocessor 96, a random access memory RAM, a read only memory ROM, and a display drive circuit 98 for the display LCD. A program 100 for processing the information required for control operations of the microprocessor 96 is stored in the read only memory ROM. The temperature of the fluid and the ambient air temperature respectively detected by the sensors S1 and S2 and the fluid temperature selected by the user are feed to the microprocessor 96 which signals the DC-to-DC converting circuit 18 to process this information to control the fluid temperature as the user desires. The comparator 98 in the heat/cool logic circuit 93 compares the user selected (set point) temperature with the fluid temperature to determine if heat should flow into or from the fluid in heat transfer contact with the side 80b as the fluid is circulated through the plumbing network PN and past the side 80b. If the comparator 98 indicates that the set point temperature is above fluid temperature, the relay 102 is in the position shown in solid lines in FIG. 7A and the side 80b causes heat to flow into the fluid. If the comparator 98 indicates that the set point temperature is below the fluid temperature, a coil 104 is energized and the relay 102 is moved to the position shown in dotted lines. This reverses the polarity of the voltage being applied to the side 80b, causing heat to flow from the fluid.

The safety board 92 logic circuit 106 as shown in FIG. 7B includes a pair of comparators 108a and 108b having their respective outputs connected to the inputs of an OR gate 110. The output of the OR gate 110 is connected to one input 112a of another OR gate 112. The signals from the sensors S4 and S3 are respectively connected to additional inputs 112b and 112c of the OR gate 112. Unless the signals at all three inputs 112a, 112b, 112c indicate that a safe condition exists at each input, the output of the OR gate 112 applies a signal through a buffer 114 to the light 97 indicating that an unsafe condition exits and the Relay 1 power is not supplied to the thermoelectric unit 16 and the pump 14. If there is no fault, a signal is applied to the Relay 1 through the buffer 114a. Thus, (a) if the temperature sensor S1A detects that temperature of the heat transfer fluid is not within predetermined limits, the thermoelectric unit 16 and pump 14 are turned off, or (b) if the level sensor S3 detects that the control unit CU is tipped over, the thermoelectric unit 16 and pump 14 are turned off, or (c) if the sensor S4 detects insufficient heat transfer fluid in the reservoir R, the thermoelectric unit 16 and pump 14 are turned off. When the signals at all three inputs 112a, 112b, and 112c indicate that a safe condition exists, the output of the OR gate 112 signals the coil 91 to be energized to close the Relay 1 and supply electrical power the thermoelectric unit 16 and the pump 14, thereby providing a failsafe mechanism.

The microprocessor 96 is programmed to interact with the user interface II (FIG. 2B) in accordance the program outlined in the process flow chart shown in FIG. 7C. The chart shows the set point temperature of the heat transfer fluid selected by the user pushing the button B1 or B2. The selected temperature is displayed for a predetermined time period (a few seconds) after which the actual temperature of the fluid is normally displayed.

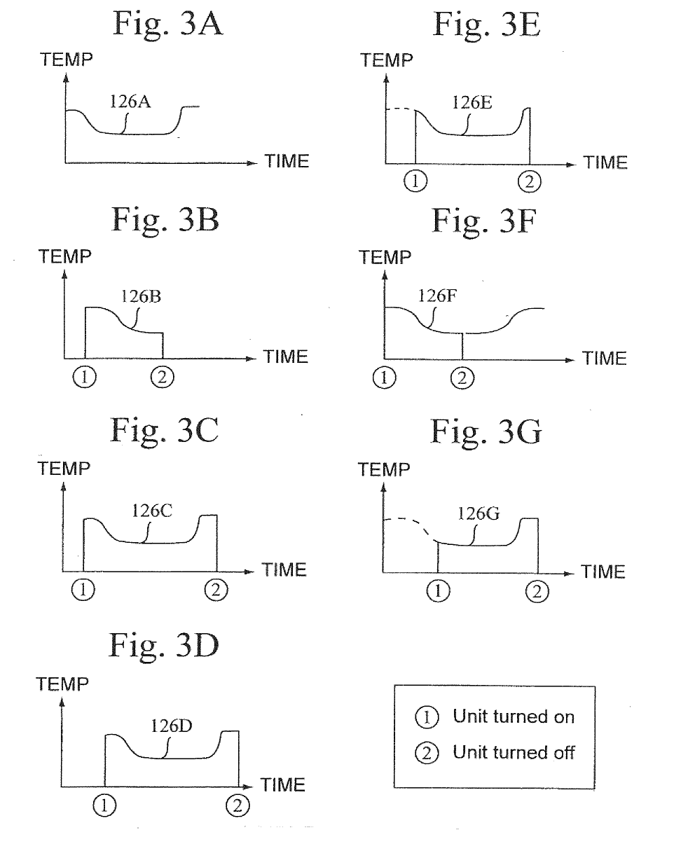

In an alternate embodiment similar to that depicted in FIG. 1A, the set point temperature may be varied in accordance with a temperature-time profile custom designed by the user. In this embodiment the user interface III is used that includes a bank of potentiometers 118 with sliders 120 that enable the user to select a plurality of different temperature of the fluid over a prolonged time period, for example, 8 hours. A selection dial 124 may be used to select the time interval between temperature changes, for example, 1 minute, 5 minutes, 10 minutes, and 1 hour. The microprocessor 96 is programmed to interact with the user interface III (FIG. 2) in accordance the program outlined in the process flow chart shown in FIG. 7D. The chart shows the set point temperatures of the heat transfer fluid selected by the user and the time intervals the user selected. As FIGS. 3A through 3G illustrate a wide variety of temperature-time profiles 126a through 126g respectively may be provided using interface III. Thus, the interface III and associated circuitry enables the user to program the control unit CU to provide an individualized custom temperature profile of the heat transfer fluid over a selected period of time including adjusting the individualized custom temperature profile to operate over portions of the selected period of time and for adjusting the duration of the selected period of time.

### Sample Specifications

1. Thermoelectric Unit 16 (TEU):
a. TEU heat transfer capacity Qₘₐₓ=150-300 Watts
b. Delta temp 60° C. or greater across the sides 80a and 80b over 24 volt range
c. Expected range of ambient temperatures for normal operation 5° C. to 50° C. (approx 41° F. to 122° F.)
d. Interfaces between TEU and heat conducting surfaces (heat sinks and heat transfer fluid) to be thermally well-coupled, using high thermal conductivity grease.

2. Water Pump 14:
a. Capable of providing at least 0.25-1 gallons per minute (GPM) at 6-20 psi.
b. Components in contact with heat transfer fluid to be oxidation and corrosion resistant and tolerate exposure water, ethylene glycol and propylene glycol for a minimum of ten years without degradation of function.

3. Fan 24-Heat Sink:
a. Fan/fins be capable of dissipating 150-300 Watts of heat and have a thermal resistance no greater than 0.15° C./Watt
b. Forced convection type heat sink, fans low noise

4. Safeties:
a. Water pump, fluid reservoir, and all tubes/vessels carrying water to be physically separated and isolated from the electronics, so that an internal leak unlikely to result in water on the electronic components.
b. A user-resetable circuit breaker to be installed which will cut power to the unit in the event of a short circuit.
c. Low water level interlock on fluid reservoir.
d. Factory settable high and low temperature interlocks on fluid temperature (not user adjustable).
e. High temperature interlock or thermal fuse on TEU to shut down the system in the event TEU temperature exceeds maximum design temperature, (typically 150° C.).
f. Tip-over detector on housing exterior

### SCOPE OF THE INVENTION

The above presents a description of the best mode we contemplate of carrying out our heating and cooling pad, control unit therefor, heating and cooling system, and method of controlling temperature while in bed, and of the manner and process of making and using them, in such full, clear, concise, and exact terms as to enable any person skilled in the art to which it pertains to make and use our pad, control unit therefor, system, and method. Our pad, control unit therefor, system, and method are, however, susceptible to modifications and alternate constructions from the illustrative embodiments discussed above which are fully equivalent. Consequently, it is not the intention to limit our pad, control unit therefor, system, and method to the particular embodiments disclosed. On the contrary, our intention is to cover all modifications and alternate constructions coming within the spirit and scope of our pad, control unit therefor, system, and method as generally expressed by the following claims, which particularly point out and distinctly claim the subject matter of our invention:

1. A personal, portable heating and cooling system for an individual comprising
a thin and flexible pad having an internal chamber for holding a heat transfer fluid and an inlet and outlet connected to the chamber,
said chamber partitioned to form therein a passageway that directs a heat transfer fluid to flow along a circuitous path between the inlet and outlet, said pad comprising a pair of plastic sheets welded together by a plurality of spot welds arranged in a predetermined pattern to form upon the pad being horizontally orientated a plurality of cushion pillows along the passageway as the heat transfer fluid flows through the pad at a pressure substantially from 3 to 20 pounds per square inch, and
a control unit having a housing enclosing a reservoir for holding the heat transfer fluid, a pump for pumping the heat transfer fluid between the reservoir and the internal chamber of the pad, and a heating and cooling apparatus for regulating the temperature of a heat transfer fluid flowing between the reservoir and the chamber.

2. The system of claim 1 where the pad has a length substantially from 12 to 48 inches and a width substantially from 12 to 36 inches and a thickness does not exceed ½ inch when the heat transfer fluid flowing through the chamber at said pressure.

3. The system of claim 2 where the chamber has a volume substantially from 100 to 2 liters with the heat transfer fluid flowing through the chamber at said pressure.

4. The system of claim 3 where the pad has a substantially rectangular configuration.

5. A personal, portable heating and cooling system for an individual comprising
a thin and flexible pad having a surface comprising of antimicrobial material and an inlet and outlet connected to an internal chamber for holding a heat transfer fluid that flows between the inlet and outlet, said heat transfer fluid flowing through the pad at a pressure substantially from 3 to 20 pounds per square inch,
a control unit having a housing enclosing a reservoir for holding the heat transfer fluid, a pump for pumping the heat transfer fluid between the reservoir and the internal chamber of the pad, and a thermoelectric unit having a heating mode and cooling mode and through which the heat transfer fluid flows upon being circulated by the pump between the inlet and outlet of the chamber, and
a control circuit for operating the pump and the thermoelectric unit, said control circuit including
a user interface having a selection member that enables the user to select a set point temperature of the heat transfer fluid flowing through the chamber,
a temperature sensor for detecting the temperature of the heat transfer fluid, a switching circuit that switches the thermoelectric unit between the heating mode and cooling mode as determined by a comparison between the set point temperature and the temperature of the heat transfer fluid as detected by the temperature sensor, and
a variable voltage power supply having a power rating substantially from 200 to 400 Watts over a 24 volt range, and the thermoelectric unit having a power rating substantially from 150 to 300 Watts over a 24 volt range, and
said pad being connected to the control unit by insulated tubing.

6. The system of claim 5 where
the pad has a length substantially from 12 to 48 inches and a width substantially from 12 to 36 inches and a thickness does not exceed ½ inch when the heat transfer fluid flowing through the chamber at said pressure, and
the chamber has a volume substantially from 100 to 2 liters with the heat transfer fluid flowing through the chamber at said pressure, and

7. The system of claim 5 including means for detecting an abnormal and unsafe condition, and upon detection of such condition, turning off the heating and cooling apparatus and providing a signal to the user that an abnormal and unsafe condition exists.

8. A personal, portable heating and cooling system for an individual adapted for operation at ambient air temperatures substantially from 41° F. to 122° F., said system comprising
a thin and flexible pad having a surface comprising of antimicrobial material and an inlet and outlet connected to an internal chamber for holding a heat transfer fluid that flows between the inlet and outlet,
a control unit having a housing enclosing a reservoir for holding the heat transfer fluid, a pump for pumping the heat transfer fluid between the reservoir and the internal chamber of the pad, and a thermoelectric unit having a heating mode and cooling mode and through which the heat transfer fluid flows upon being circulated by the pump between the inlet and outlet of the chamber, and
a control circuit for operating the pump and the thermoelectric unit, said control circuit including
a user interface having a selection member that enables the user selected a set point temperature of the heat transfer fluid,
a microprocessor for controlling the operation of the pump and thermoelectric unit,
a first temperature sensor for detecting ambient air temperature,
a second temperature sensor for detecting the temperature of the heat transfer fluid, and
a comparator to compare the user selected set point temperature with the temperature detected second temperature sensor and, as required based on said comparison, signal the microprocessor to operate the thermoelectric unit to increase or decrease the temperature of the heat transfer fluid flowing between the reservoir and the chamber,
said microprocessor controlling the rate of increase or decrease in temperature in response to the ambient air temperature detected by the first temperature sensor.

9. A personal, portable heating and cooling system for an individual comprising
a thin and flexible pad having an internal chamber for holding a heat transfer fluid and an inlet and outlet connected to the chamber,
said chamber partitioned to form therein a passageway that directs a heat transfer fluid to flow along a circuitous path between the inlet and outlet,
a control unit having a housing enclosing a reservoir for holding the heat transfer fluid, a pump for pumping the heat transfer fluid between the reservoir and the internal chamber of the pad, and a heating and cooling apparatus for regulating the temperature of a heat transfer fluid flowing between the reservoir and the chamber,
a temperature sensor for detecting the temperature of the heat transfer fluid, said heating and cooling apparatus being turned off when the temperature sensor detects that temperature of the heat transfer fluid is not within predetermined limits,
a level sensor on the exterior of the control unit for detecting the spatial orientation of the control unit, said heating and cooling apparatus and pump being turned off when the level sensor detects that the control unit is tipped over, and
a fluid sensor for detecting the amount of heat transfer fluid in the reservoir, said heating and cooling apparatus and pump being turned off when the fluid sensor detects insufficient heat transfer fluid in the reservoir.

10. The system of claim 9 including means for selecting the set point temperature of the heat transfer fluid in the reservoir and displaying said selected set point temperature for a predetermined time period after which the actual temperature of the fluid is displayed.

11. The system of claim 9 including means for enabling the user to program the heating and cooling apparatus to provide an individualized custom temperature profile of the heat transfer fluid over a selected period of time.

12. The system of claim 11 including means for adjusting the individualized custom temperature profile to operate over portions of the selected period of time and means for adjusting the duration of the selected period of time.

13. A personal, portable heating and cooling system for an individual comprising
pad having a surface comprising of antimicrobial material and including an internal liquid-tight chamber having an inlet and an outlet, and
a portable, compact control unit adapted to be placed in fluid communication with the chamber,
said control unit including a housing having a reservoir for holding a heat transfer fluid, a fluid plumbing network that circulates the heat transfer fluid at a pressure substantially from 3 to 20 pounds per square inch between the chamber and the reservoir upon placing the pad outlet in fluid communication with the reservoir and the reservoir in fluid communication with the pad inlet, a bi-directional heat pump that cools or heats the heat transfer fluid as said heat transfer fluid is circulated, and a user interface for controlling the temperature of the heat transfer fluid as it flows through the chamber of the pad.

14. A personal, portable heating and cooling system for an individual comprising
a thin and flexible pad having an internal chamber for holding a heat transfer fluid and an inlet and outlet connected to the chamber,
said chamber partitioned to form therein a passageway that directs a heat transfer fluid to flow along a circuitous path between the inlet and outlet,
a control unit having
a housing enclosing a reservoir for holding the heat transfer fluid,
a pump for pumping the heat transfer fluid between the reservoir and the internal chamber of the pad,
a heating and cooling apparatus for regulating the temperature of a heat transfer fluid flowing between the reservoir and the chamber,
a power supply that provides a constant output voltage substantially from 0 to 24 volts, and
a converting circuit coupled to the power supply that is interactive active with a user interface, said converting circuit having an operator element that enables a user to set the temperature of the fluid by converting the constant output voltage to a selectable variable voltage.

15. The system of claim 16 where the converting circuit provides a variable voltage from 0 to 24 volts and the power supply has a power rating substantially from 200 to 400 Watts over a 24 volt range, and the heating and cooling apparatus has a power rating substantially from 150 to 300 Watts over a 24 volt range.

16. A personal, portable heating and cooling system for an individual adapted for operation in an ambient air environment at temperatures substantially from 41° F. to 122° F., said system comprising
a thin and flexible pad having a surface comprising of antimicrobial material and having an internal chamber for holding a heat transfer fluid and an inlet and outlet connected to the chamber,
said chamber partitioned to form therein a passageway that directs a heat transfer fluid to flow between the inlet and outlet, and
a control unit having a housing enclosing a reservoir for holding the heat transfer fluid, a pump for pumping the heat transfer fluid between the reservoir and the internal chamber of the pad, and a thermoelectric unit having a first side in heat transfer contact with the ambient air environment and a second side,
said outlet of the pad being in communication with the thermoelectric unit so fluid flowing from the pad passes the second side and makes heat transfer contact with said second side,
said thermoelectric unit being responsive to alternating a polarity of an applied voltage so heat flows from said second side into the fluid passing thereby or heat flows from the fluid passing thereby into the second side,
said control unit including an electronic control circuit having a user interface that enables a user to select the temperature of the fluid and compares said selected temperature with the temperature of the fluid and alternates the polarity of the applied voltage until the temperature of the fluid is substantially equal the selected temperature.

17. A thin and flexible heating and cooling pad comprising
a pair of liquid impenetrable flexible plastic sheets welded together to form between said sheets a circuitous passageway, and
an inlet and an outlet in fluid communication with the passageway,
said passageway including a plurality of cushion pillows as the heat transfer fluid flows through the pad upon the pad being horizontally orientated and inflated with a heat transfer fluid at a pressure substantially from 3 to 20 pounds per square inch.

18. The pad of claim 16 where each sheet is substantially rectangular, each sheet has substantially the same dimensions, the sheets each have edges and are bonded along the edges to form the overall width and length dimensions of the chamber, which are only slightly less than the width and length dimensions of the sheets, and said sheets each having a surface comprising of antimicrobial material.

19. The pad of claim 18 having a length substantially from 12 to 48 inches, a width substantially from 12 to 36 inches

20. The pad of claim 17 where the inlet and outlet are nearby each other.

21. A thin and flexible heating and cooling pad comprising
a pair liquid impenetrable flexible plastic sheets bonded together to form between them an internal chamber for holding a heat transfer fluid and an inlet and outlet in communication with the chamber, said pad having a maximum thickness of substantially ½ inch when said chamber is filled with the heat transfer fluid,
said sheets overlying each other and bonded together by (a) linear welds between facing surfaces of said overlying sheets to form within the chamber a passageway that directs a heat transfer fluid to flow along a circuitous path between the inlet and outlet, and (b) spot welds within the passageway to form fluid mixing zones along the passageway.

22. The pad of claim 20 where the number of spot welds per square inch of facing surfaces is substantially from 4 to 25.

23. The pad of claim 20 where the area of spot welds is substantially from 0.003 to 0.012 square inches.

24. The pad of claim 20 where the spot welds are arranged in a predetermined grid pattern comprising intersecting grid lines with individual spot welds located at intersections of the grid lines.

25. A control unit for a heating and cooling pad comprising
a housing containing a reservoir for holding a heat transfer fluid, a temperature sensor for detecting the temperature of the heat transfer fluid, a pump for pumping the heat transfer fluid, a heating and cooling apparatus for regulating the temperature of a heat transfer fluid, and a control unit,
said control unit including an electronic controller having a user interface on an exterior of the housing that enables a user to set the temperature of the fluid,
said electronic controller including a comparator to compare a user selected set point temperature with the temperature detected by temperature sensor and, if required based on said comparison, signal the heating and cooling apparatus to increase or decrease the temperature of the heat transfer fluid.

26. The control unit of claim 25 including a temperature sensor for detecting the temperature of the heat transfer fluid, said heating and cooling apparatus being turned off when the temperature sensor detects that temperature of the heat transfer fluid is not within predetermined limits.

27. The control unit of claim 25 including a level sensor for detecting the spatial orientation of the control unit, said heating and cooling apparatus being turned off when the level sensor detects that the control unit is tipped over.

28. The control unit of claim 25 including a fluid sensor for detecting the amount of heat transfer fluid in the reservoir, said heating and cooling apparatus being turned off when the fluid sensor detects insufficient heat transfer fluid in the reservoir.

29. The control unit of claim 25 including means for detecting an abnormal and unsafe condition and upon detection turning off the heating and cooling apparatus and providing a signal to the user that an abnormal and unsafe condition exists.

30. The control unit of claim 25 including means for selecting the set point temperature of the heat transfer fluid in the reservoir and displaying said selected set point temperature for a predetermined time period after which the actual temperature of the fluid is displayed.

31. The control unit of claim 25 including means for enabling the user to program the heating and cooling apparatus to provide an individualized custom temperature profile of the heat transfer fluid over a selected period of time

32. The control unit of claim 25 including means for adjusting the individualized custom temperature profile to operate over portions of the selected period of time.

33. The control unit of claim 32 including means for adjusting the duration of the selected period of time.

34. The control unit of claim 25 including a fan and a radiator device for facilitating heat transfer.

35. A control unit for a heating and cooling pad comprising
a housing containing a reservoir for holding a heat transfer fluid, a pump for pumping the heat transfer fluid, a heating and cooling apparatus for regulating the temperature of a heat transfer fluid, and a control unit including
a power supply that provides a constant output voltage substantially from 0 to 24 volts, and
a converting circuit coupled to the power supply that is interactive active with a user interface,
said converting circuit having an operator element that enables a user to set the temperature of the fluid by converting the constant output voltage to a selectable variable voltage.

36. A method of controlling temperature while in bed comprising
(a) positioning on the bed an enlarged thin flexible heating and cooling pad having an internal chamber holding a heat transfer fluid, said pad having width and length dimensions sufficient so a substantial portion of a user's body contacts the pad directly or indirectly when lying on the pad, said pad having a maximum height of substantially ½ inch when the chamber is filled with the heat transfer fluid at a pressure substantially from 3 to 20 pounds per square inch,
(b) connecting the pad to a control unit having a housing enclosing a reservoir for holding the heat transfer fluid, a pump for pumping the heat transfer fluid between the reservoir and the pad, and a heating and cooling apparatus for regulating the temperature of a heat transfer fluid flowing between the reservoir and the chamber, said control unit including an electronic controller having a user interface that enables a user enables a user to increase or decrease the temperature of the fluid, and
(c) increasing or decreasing the temperature of the heat transfer fluid flowing through the pad.
